(19) United States
(12) Patent Application Publication
(10) Pub. No.: US 2018/0303793 A1
(43) Pub. Date: Oct. 25, 2018

(54) METHODS AND COMPOSITIONS FOR TREATING TRAUMATIC BRAIN INJURY

(71) Applicant: SCYTHIAN BIOSCIENCES INC., S.W. Calgary (CA)

(72) Inventor: David A. Schrader, New York, NY (US)

(21) Appl. No.: 15/767,706

(22) PCT Filed: Oct. 17, 2016
(86) PCT No.: PCT/US2016/057304

§ 371 Date: Apr. 12, 2018

Related U.S. Application Data
(60) Provisional application No. 62/242,457, filed on Oct. 16, 2015.

(51) Int. Cl. A61K 31/352; A61P 25/00

(52) U.S. Cl. CPC A61K 31/352; A61K 45/06; A61K 2300/00; A61P 25/00

(57) ABSTRACT

Methods and compositions for treating traumatic brain injury in a subject are provided.

This patent application claims the benefit of priority from U.S. Provisional Patent Application Ser. No. 62/242,457, filed Oct. 16, 2015, the content of which is hereby incorporated by reference in its entirety.

### BACKGROUND

Traumatic brain injury (TBI) occurs when an external mechanical force causes brain dysfunction.

Traumatic brain injury usually results from a violent blow or jolt to the head or body. An object penetrating the skull, such as a bullet or shattered piece of skull, can also cause traumatic brain injury.

TBI has been called the signature injury of modern warfare. A Rand Corporation survey published in 2008 estimated that as many as 19.5% of those who deployed to Iraq or Afghanistan sustained TBI (RAND; Reports and Bookstore; Research Briefs; RB-9336; Invisible Wounds: Mental Health and Cognitive Care Needs of America's Returning Veterans). The Defense Centers of Excellence for Psychological Health and Traumatic Brain Injury (DCOE) estimates that 1.7 million people a year suffer a TBI and reports that almost 25,000 military members last year were diagnosed with a new TBI (dcoe with the extension .health.mil of the world wide web).

Traumatic brain injury can be caused by a variety of insults. In the operations in Southwest Asia the vast majority of mild TBI (mTBI) has been caused by blast exposure. mTBI may cause temporary dysfunction of brain cells. More serious traumatic brain injury can result in bruising, torn tissues, bleeding and other physical damage to the brain that can result in long-term complications or death.

A concussion is a type of traumatic brain injury that is caused by a blow to the head or body, a fall, or another injury that jars or shakes the brain inside the skull. Symptoms of a concussion range from mild to severe and can last for hours, days, weeks, or even months. Repeated concussions or a severe concussion may lead to long-lasting problems with movement, learning, or speaking. Rest, coupled with acetaminophen for any headache pain, is currently considered the most appropriate way to allow the brain to recover from a concussion.

While the sequelae of serious brain injury have been studied for many years and are relatively well characterized, the sequelae of blast-induced or concussive mTBI require better understanding. Current literature suggests that the majority of symptoms are neurosensory in nature (Hoffer et al. Otol Neurotol. 2010 31(2):232-6; Hoffer et al. PLoS ONE 2013 8(1): e54163. doi:10.1371/journal.pone.0054163). Dizziness, hearing loss, headaches, and neurocognitive dysfunction are frequent sequelae and, while these resolve over time in a certain portion of the population, they persist and can worsen in many individuals (Hoffer et al. Otol Neurotol. 2010 31(2):232-6). Moreover, there is an emerging body of literature to suggest that over time mTBI secondary to blast can exhibit characteristics that suggest the presence of neurodegenerative disorders in these patients (Woods et al. ACS Chem. Neurosci. 2013 Epub ahead of press. DOI: 10.1021/cn300216h).

Given the role of N-methyl-D-aspartate (NMDA), α-amino-3-hydroxy-5-methyl4-isoxazole proprionate (AMPA) and kainite these receptors in neurological damage, research efforts have focused on using antagonists to these receptors to interfere with the receptor mediated calcium efflux leading to cellular death and necrosis.

Some of the research on these antagonists has focused on cannabinoids, a subset of which are NMDA receptor antagonists. See, for example, U.S. Pat. Nos. 5,538,993, 5,521,215, and 5,284,867.

However, the NMDA receptor antagonist dexanabinol was tested in Phase II and Phase III clinical trials (Shohami E & Biegon A. CNS Neurol Disord Drug Targets. 2014; 13(4):567-73). In prior studies of dexanabinol for use in the treatment of concussion, one study showed that it lowered intracranial pressure. However, other studies appeared inconclusive at best and found that it did not inhibit gliosis and the subsequent immune cascade. The studies failed to provide adequate support for the use of dexanabinol as a single agent for the treatment of concussion.

U.S. Pat. No. 6,630,597 proposes use of cannabinoids with substantially no binding to the NMDA receptor as a neuroprotectant.

Cannabinoids as a possible treatment for concussions has also been disclosed by Shohami et al. (Journal of Neurotrauma 2009, 10(2): 109-119. doi:10.1089/neu.1993.10.109) Fernández-Ruiz et al. (Handb Exp Pharmacol. 2015; 231:233-59) and Pryce et al. (Handb Exp Pharmacol. 2015; 231:213-31)

The cannabinoid CB2 receptor as also been disclosed as a target for inflammation-dependent neurodegeneration (Ashton J C & Glass M. Current Neuropharmacology. 2007; 5(2):73-80).

More recently, progesterone treatment was being studied as a possible agent to improve cognitive outcome in TBI (Si et al. Neurosci Lett. 2013 Aug. 14. pii: S0304-3940(13)00714-3. doi: 10.1016/j.neulet.2013.07.052; Baykara et al. Biotech Histochem. 2013 July; 88(5):250-7. doi: 10.3109/10520295.2013.769630; and Meffre et al. Neuroscience. 2013 Feb. 12; 231:111-24. d10.1016/j.neuroscience.2012.11.039).

Identification of therapeutic agents and methods for their use in preventing or treating the neurosensory deficits associated with blast or concussion induced mTBI is needed.

### SUMMARY

The present invention relates to methods and compositions for treating traumatic brain injury in a subject.

An aspect of the present invention relates to a method for treating traumatic brain injury in a subject which comprises administering to the subject a first composition comprising an N-Methyl-D-aspartate (NMDA) receptor antagonist and a second composition comprising an anti-inflammatory agent capable of crossing the blood brain barrier.

Another aspect of the present invention relates to a method for treating traumatic brain injury in a subject which comprises administering to the subject a first composition comprising an N-Methyl-D-aspartate (NMDA) receptor antagonist and a second composition comprising a CB2 agonist, an agent which effectively increases an endogenous CB2 agonist and/or an agent which modifies levels of anandamide (AEA) or 2-arachidonoyl glycerol (2-AG).

In one nonlimiting embodiment, the first composition administered comprises 7-hydroxy-delta6-tetrahydrocannabinol 1,1-dimethylheptyl.

In one nonlimiting embodiment, the second composition comprises a non-cannabinoid CB2 agonist.

In another nonlimiting embodiment, the second composition comprises a cannabinoid CB2 agonist that also binds to an NMDA receptor.

In another nonlimiting embodiment, the second composition comprises an agent which increases levels of AEA.

In another nonlimiting embodiment, the second composition comprises an agent that decreases levels of 2-AG.

In yet another nonlimiting embodiment, the second composition comprises an inhibitor of fatty acid amide hydrolase.

In one nonlimiting embodiment, the traumatic brain injury treated is concussion.

Yet another aspect of the present invention relates to compositions for treatment of traumatic brain injury. In one nonlimiting embodiment, the composition comprises an N-Methyl-D-aspartate (NMDA) receptor antagonist and an anti-inflammatory agent capable of crossing the blood brain barrier. In one nonlimiting embodiment, the composition comprises an N-Methyl-D-aspartate (NMDA) receptor antagonist and a CB2 agonist, an agent which effectively increases an endogenous CB2 agonist and/or an agent which modifies levels of anandamide (AEA) or 2-arachidonoyl glycerol (2-AG).

### DETAILED DESCRIPTION

The present invention provides methods for treating traumatic brain injury in a subject. In one nonlimiting embodiment, the traumatic brain injury treated is concussion.

The methods of the present invention comprise administering to a subject suffering from a traumatic brain injury a first composition comprising an N-Methyl-D-aspartate (NMDA) receptor antagonist.

By “NMDA receptor antagonist” as used herein, it is meant to include the class of agents that work to antagonize or inhibit the action of N-Methyl-D-aspartate receptor (NMDA). Examples include, but are not limited to, dizocilpine (MK-801), ketamine, memantine, phencyclidine, gascyclidine, AP5, amantadine, ibogaine, nitrous oxide riluzole, dextrorphan, AP-7,

tiletamine, midafotel, aptiganel and 7-hydroxy-delta6-tetrahydrocannabinol 1,1-dimethylheptyl (dexanabinol: HU-211). In one nonlimiting embodiment, the NMDA receptor antagonist is a noncompetitive NMDA receptor antagonist such as dexanabinol, GK-11 or gascyclidine, or phencyclidine or an uncompetitive NMDA receptor antagonist such as dizocilpine. Additional nonlimiting examples of NMDA receptor antagonists useful in the present invention are disclosed in U.S. Pat. No. 5,521,215, teachings of which are incorporated herein by reference in their entirety. In one nonlimiting embodiment, the NMDA receptor antagonist is 7-hydroxy-delta6-tetrahydrocannabinol 1,1-dimethylheptyl (Dexanabinol: HU-211).

In one nonlimiting embodiment, the first composition is administered via a regimen effective to inhibit swelling which occurs from the traumatic brain injury.

In one nonlimiting embodiment, the first composition is administered within 12 hours of the traumatic brain injury, or alternatively with 6 hours of the traumatic brain injury, or alternatively within 3 hours of the traumatic brain injury. In these embodiments, the first composition may be administered as a single dose or as multiple doses.

In one nonlimiting embodiment, multiple doses of the first composition are administered over a 72 hour period following the traumatic brain injury.

In one nonlimiting embodiment, the first composition is administered daily or every two days until symptoms of the traumatic brain injury are alleviated.

The first composition may be administered by any route providing for delivery of effective amounts of the NMDA receptor antagonist to the brain. Examples of routes of administration include, but are in no way limited to, intravenous, intranasal, oral, topical, transdermal or via inhalation.

As will be understood by the skilled artisan upon reading this disclosure, dosages can be determined by the attending physician, according to the extent of the injury to be treated, method of administration, patient's age, weight, contraindications and the like. Nonlimiting examples of dosages include a single i.v. dose of 150 mg or greater, and doses in the range of from 0.05 mg to about 50 mg per kg body weight, in a regimen of 1-4 times a day or every other day.

In one nonlimiting embodiment, the method of the present invention further comprises administration to the subject of a second composition comprising an anti-inflammatory agent capable of crossing the blood brain barrier. The anti-inflammatory agent can be administered in accordance with any dosing regimen effective to inhibit inflammation of the brain resulting from the traumatic injury. The anti-inflammatory agent can be administered before, simultaneously or after administration of the NMDA receptor antagonist.

In one nonlimiting embodiment, the method of the present invention further comprises administration to the subject of a second composition comprising a CB2 agonist, an agent which effectively increases an endogenous CB2 agonist and/or an agent which modifies levels of anandamide (AEA) or 2-arachidonoyl glycerol (2-AG).

By “CB2 agonist” as used herein, it is meant to include classes of agents which activate the cannabinoid 2 receptor in a selective or nonselective manner. In one nonlimiting embodiment, the CB2 agonist is a non-cannabinoid CB2 agonist. In another nonlimiting embodiment, the CB2 agonist is a cannabinoid CB2 agonist that also binds to an NMDA receptor.

In one nonlimiting embodiment, the second composition comprises cannabidiol (CBD), a naturally occurring chemical in certain varieties of marijuana. CBD has no psychoactive effect. CBD acts as a CB-2 agonist and presents a broad range of anti-inflammatory and immune inhibitory effects.

Alternatively, or in addition, the second composition may comprise an agent which effectively increases an endogenous CB2 agonist and/or an agent which modifies levels of anandamide (AEA) or 2-arachidonoyl glycerol (2-AG). In one nonlimiting embodiment, the second composition comprises an agent which increases levels of AEA. In another nonlimiting embodiment, the second composition comprises an agent which decreases levels of 2-AG. In one nonlimiting embodiment, the second composition comprises an inhibitor of fatty acid amide hydrolase. In one nonlimiting embodiment, the second composition comprises AEA.

In one nonlimiting embodiment, the second composition is administered via a regimen effective to inhibit gliosis which occurs from the traumatic brain injury.

The second composition can be administered before, simultaneously or after administration of the first composition.

In one nonlimiting embodiment, the first and second compositions are formulated together into a single composition comprising both therapeutic agents.

In one nonlimiting embodiment, the second composition is administered 12 to 72 hours following the traumatic brain injury.

In one nonlimiting embodiment, the second composition is administered within 12 hours of the traumatic brain injury, or alternatively with 6 hours of the traumatic brain injury, or alternatively within 3 hours of the traumatic brain injury. In these embodiments, second composition may be administered as a single dose or as multiple doses.

In one nonlimiting embodiment, the second composition is administered with the first composition as a single composition within 12 hours of the traumatic brain injury, or alternatively with 6 hours of the traumatic brain injury, or alternatively within 3 hours of the traumatic brain injury. In these embodiments, the composition comprising the first and second compositions may be administered as a single dose or as multiple doses.

In one nonlimiting embodiment, the second composition is administered daily for up to 7 days following the traumatic brain injury.

In one nonlimiting embodiment, the second composition is administered daily or every other day until symptoms of the traumatic brain injury are alleviated.

The second composition may be administered by any route providing for delivery of effective amounts of the CB2 agonist, the agent which effectively increases an endogenous CB2 agonist and/or the agent which modifies levels of anandamide (AEA) or 2-arachidonoyl glycerol (2-AG) to the brain. Examples of routes of administration include, but are in no way limited to, intravenous, intranasal, oral, topical, transdermal or via inhalation.

As will be understood by the skilled artisan upon reading this disclosure, dosages can be determined by the attending physician, according to the extent of the injury to be treated, method of administration, patient's age, weight, contraindications and the like.

Also provided are pharmaceutical compositions for treatment of traumatic brain injury. In one nonlimiting embodiment, the composition comprises an N-Methyl-D-aspartate (NMDA) receptor antagonist and an anti-inflammatory agent capable of crossing the blood brain barrier as well as a pharmaceutically acceptable vehicle. In one nonlimiting embodiment, the pharmaceutical composition comprises an N-Methyl-D-aspartate (NMDA) receptor antagonist and a CB2 agonist, an agent which effectively increases an endogenous CB2 agonist and/or an agent which modifies levels of anandamide (AEA) or 2-arachidonoyl glycerol (2-AG) as well as a pharmaceutically acceptable vehicle.

As used herein “pharmaceutically acceptable vehicle” includes any and all solvents, excipients, dispersion media, coatings, antibacterial and antifungal agents, isotonic and absorption delaying agents, and the like which are compatible with the activity of the therapeutic compositions and are physiologically acceptable to a subject. An example of a pharmaceutically acceptable vehicle is buffered normal saline (0.15 M NaCl). The use of such media and agents for pharmaceutically active substances is well known in the art. Except insofar as any conventional medium or agent is incompatible with the therapeutic composition, use thereof in the compositions suitable for pharmaceutical administration is contemplated. Supplementary active compounds can also be incorporated into the compositions.

Dispersions comprising the therapeutic compositions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and oils (e.g. vegetable oil). The proper fluidity can be maintained, for example, by the use of a coating such as lecithin, by the maintenance of the required particle size in the case of dispersion, and by the use of surfactants.

Sterile injectable solutions can be prepared by incorporating the therapeutic compositions in the required amount in an appropriate solvent with one or a combination of ingredients enumerated above, as required, followed by filter sterilization. Generally, dispersions are prepared by incorporating the therapeutic compositions into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) optionally plus any additional desired ingredient from a previously sterile-filtered solution thereof.

Solid dosage forms for oral administration include ingestible capsules, tablets, pills, lollipops, powders, granules, elixirs, suspensions, syrups, wafers, buccal tablets, troches, and the like. In such solid dosage forms the active compounds are mixed with at least one inert, pharmaceutically acceptable excipient or diluent or assimilable edible vehicle such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, or incorporated directly into the subject's diet. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The percentage of the therapeutic compounds in the compositions and preparations may, of course, be varied. The amount of the therapeutic compounds in such therapeutically useful compositions is such that a suitable dosage will be obtained.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

The pharmaceutical compositions of therapeutic compounds can also be administered in time-release or depot form, to obtain sustained release of the therapeutic compounds over time. The therapeutic compounds of the invention can also be administered transdermally (e.g., by providing the therapeutic compound, with a suitable vehicle, in patch form).

A concussion causes damage to the brain in various steps. First, there is the initial impact that causes tissue damage. Second, the brain tissue begins to swell through inflammation resulting in an increase in intracranial pressure. Because the brain is in a fixed area surrounded by the skull, as the brain expands with inflammation, it presses against the skull with increasing pressure and causes extensive additional damage. This inflammation starts within the first few hours after the initial injury. A third wave of damage occurs as a result of a process called gliosis which starts within 6-12 hours after the initial injury when a cascade of immune responses takes place in the brain tissue causing the infiltration of white blood cells (leukocytic and macrophage infiltration) and the release of cytokines. This immune cascade itself is the cause of extensive tissue injury.

By working on two different receptors in this process, the combination therapies of the present invention are expected to prevent the inflammation and to inhibit gliosis and the associated immune cascade, thus providing a useful therapy for concussion as well as other traumatic brain injury.

The following nonlimiting examples are provided to further illustrate the present invention.

### EXAMPLES

### Example 1: Rodent Studies

The ability of a combination therapy comprising the selected NMDA antagonist dexanabinol (also known as HU 211) and the CB2 agonist cannabidiol (CBD) (combination therapy referred to herein as SP-treated) to reduce the neurosensory sequelae of blast induced or concussive mTBI as compared to placebo (non-treated) is confirmed in a small rodent model. These studies are demonstrative of the ability of this combination therapy to reduce damage to the brain and the hearing and balance sensory organs of blast induced mTBI in a small rodent model when compared to placebo when the medicine is administered after the blast or concussion. In addition, histological differences are identified in SP-treated vs. non-treated animals. Any difference in lipodemic characteristics in SP-treat vs. non-treated animals are also assessed.

There are 8 initial treatment groups of 15 rats per group/TBI model. The three TBI models are blast exposure (BLAST), a weight-drop closed head injury model (CHI), and a fluid pressure injury model (FPI). The groups are: Group A (SP alone), Group B (Vehicle alone), Group C (Blast+SP), Group D (BLAST+Vehicle), Group E (CHI+SP), Group F (CHI+vehicle), Group G (FPI+SP), and Group H (FPI+vehicle). Use of 15 rats per group allows for examination of short and longer-term outcomes. All groups will undergo behavioral testing for sensorimotor, cognitive, and working memory function, hearing testing, and balance testing pre-exposure and at 3 and 7 days post-exposure. One third of each group of animals will be sacrificed at seven days (5 per group) and will undergo a gross examination and series of histochemical tests. The second group of five animals per group will undergo behavioral and hearing testing at day 14, 21, and 31 days before being sacrificed at day 31 at which point they will undergo the same terminal analysis as the seven day animals. The final five animals will be the long term group and will undergo behavioral and hearing tests at day 3, day 7, day 31, day 61, and day 91 at which point they will be sacrificed for the same histological analysis as the first two time point groups.

### Example 2: Blast Exposure Model (BLAST)

Rodent Blast Wave Tube Exposures: The shock wave generator system is composed of a three foot compression tube that makes a pressure-sealed fit by aligning with a single, 12 foot long by 8 inch inner diameter condensing tube. The compression tube is charged by an industrial compressor. A hydraulic actuating device presses a sliding portion of the condensing tube against the compression tube over a plastic film to seal off the compression tube. The system is operated remotely for management of the compression chamber to a selected pressure, moving new film in position, closure of the condensing tube to the compression tube to make a air-tight seal over the non-burst film, activation of an internal armature to burst the film at selected pressures, and moving new non-burst film into place for the next blast event. The output of the tube renders a supersonic, Friedlander type overpressure/underpressure wave which simulates the shock pressure wave from explosive detonations. Overpressure intensities can be generated from 2 pounds per square inch (psi) or 14.5 kilopascals (kPa) to >50 psi (0.35 megapascals). Exposures for the single blast overpressure (BOP) studies will range between 10 and 20 psi (depending on the initial reactions as determined on refinement animals). All animals will be anesthetized and placed in an animal holding tube inserted and secured one-foot within the end of the condensing tube. The animal holding tube positions the animal with the rat's dorsal head surface to the on-coming shock wave. Subjects will be positioned 10 feet from the tube film diaphragm and will receive a BOP wave in a head-on orientation. The holding tube allows for Isoflurane gas to feed to the animal to induce anesthesia allowing exposures to live but anesthetized animals. BOP waves will be measured and displayed for peak intensities, rise time and BOP wave durations using a Pacific Instruments 6000 DAQ with up to 32 channels, each with 250 kHz recording speed along with Dytran pressure transducers rated for 0 50 PSI measurement range and electronic conditioners interfaced with computers. An exposure will consist of anesthetized animals receiving a single blast wave exposure. Initial investigations examine the effects of single 10-20 psi (Friedlander wave with overpressure-underpressure sequence) which have been shown to demonstrate pathological effects (see Balaban et al J Neurosci Methods 2016 pii: S0165-0270(16)00053-4. doi: 10.1016/j.jneumeth.2016.02.001. Epub ahead of print).

### Example 3: Closed Head Injury Model (CHI)

Rats undergo mild TBI (mTBI) by methods previously described by Foda and Marmarou (J Neurosurg. 1994 80(2):301-13). Specifically, male Sprague-Dawley rats are anesthetized with 3% isoflurane, 70% N₂O and 30% O₂ in a bell jar until no response to paw or tail pinch. After initial anesthesia, animals are maintained via nose cone with a maintenance dosage of 1-2.5%. Sterile eye lubricant is used so eyes do not dry out during surgery. The animal's head is shaved and wiped down with chlorohexadine. An incision is made to expose the skull. A steel disc (10 mm in diameter and 3 mm thickness) is attached to the skull between bregma and lambda suture lines using dental acrylic. Animals are then moved onto a foam mattress (Type E polyurethane foam) underneath the trauma device. A weight of 450 grams is allowed to fall freely through a vertical tube from 1 meter. Body temperature is monitored using a rectal temperature probe and temporalis muscle temperature is monitored as an indirect measurement of brain temperature. Sham animals will undergo all surgical procedures but are not subjected to the brain trauma. After injury, animals are sutured closed and returned to their home cage and given food and water ad libitum. If the animal has difficulty eating after injury then the animal is euthanized. Animals have a tail artery catheter placed prior to brain injury and after brain trauma, animals are intubated and administered rocuronium or pancuronium as the paralytic.

### Example 4: Fluid Percussion Injury Model (PFI)

Sprague-Dawley rats are anesthetized with 3% isoflurane for induction of anesthesia in a custom built anesthesia chamber. Toe pinch is performed to make sure animals are appropriately anesthetized. Animals' respiratory rate is visually assessed. Isoflurane anesthesia is then maintained via nose cone and the injury cap is placed on the exposed dura as follows: the rat's head is shaved and swabbed with clorohexadine solution; the rat is then placed in a stereotaxic frame and the scalp surgically incised; a parasagittal craniotomy (4.8 mm) using a trephine is performed at 3.8 mm posterior to bregma and 2.5 mm lateral to the midline; a sterile plastic injury tube (the plastic connector of a sterile needle cut 1 cm in length and trimmed to fill the craniotomy perfectly) is next placed over the exposed dura and bonded by crynoacrylic adhesive to the skull; dental acrylic is then poured around the injury tube to obtain a perfect seal; after the acrylic has hardened, the injury tube is plugged with sterile gel foam sponge or with a Luer-Lok adapter, the scalp is stapled/sutured back; animals are removed from the anesthesia and returned to their home cage.

Twenty-four hours after the previous preparation, the rats are anesthetized with 3% isoflurane via a custom built anesthesia chamber and toe pinch is used to determine anesthesia level. The animal is placed on the table and anesthesia is administered via a nose cone until catheters are placed and the animals is intubated. A catheter is placed in the right femoral artery for those animals not undergoing behavioral testing or tail artery for those animals undergoing behavioral testing to monitor arterial blood pressure and blood gases. Brain temperature is indirectly measured by a thermistor placed in the left temporalis muscle and maintained at a normothermic (37° C.) level prior and subsequent to TBI. Rectal temperature is also maintained at normothermic levels. After intubation, the animal is connected to a respirator and ventilated with 0.5-1% isoflurane in a mixture of 70% nitrous oxide and 30% oxygen. The animal is paralyzed with rocuronium for mechanical ventilation to maintain arterial blood gases within normal limits. The fluid percussion (F-P) device consists of a plexiglass cylindrical reservoir bounded at one end by a rubber-covered plexiglass piston with the opposite end fitted with a transducer housing and a central injury screw adapted for the rat's skull. The entire system is filled with 37° C. isotonic saline. An aseptic metal injury screw is next firmly connected to the plastic injury tube of the intubated and anesthetized rat. The injury is induced by the descent of a metal pendulum striking the piston, thereby injecting a small volume of saline epidurally into the closed cranial cavity and producing a brief displacement (18 msec) of neural tissue. The amplitude of the resulting pressure pulse is measured in atmospheres by a pressure transducer and recorded on a PowerLab chart recording system. Sham animals will undergo all surgical procedures but are not subjected to the F-P pulse. A mild (1.4-1.6 atm) injury will be studied. Following injury, catheters are removed and the incisions are stapled/sutured closed. Anesthesia is discontinued and animals awake approximately 30 minutes after injury and are placed in an individual cage supplied with food and water ad libitum until termination of the study.

### Example 5: Post-Exposure Husbandry

All animals are monitored for respiratory rate, heart rate and blood oxygen saturation using a STARR Life Sciences, Corp., and Pulse oximetry before and after the exposure and for the entire recovery period after the exposure. If animals show signs of extreme pain or that they may not recover after blast exposure, the veterinarian will examine the animal. Animals will be closely observed for signs of pain to include vocalization, in appetence, aggression, guarding, and extreme agitation. The animal may or may not be euthanized based on the veterinarian's recommendation. To avoid potential pain reaction, ketoprofen will be given immediately after the blast exposure. A pain assessment will be applied beginning immediately after recovery from anesthesia. The major indices to assess will be: activity, physical appearance, vocalizing, grinding teeth, feeding/drinking behavior and physiological signs such as respiratory rate, heart rate and oxygen saturation). If any of the indicators suggest the animal is still experiencing pain, then higher doses will be delivered or if intractable, animals will never be left in pain and the veterinarian may decide to euthanize the rat. Pain relief will be as required. Collection of functional measures can be postponed to the next day if animals display pain levels that prevent testing. At that time, the animal's status will be reevaluated and testing may commence or again be delayed. Once functional and cognitive measures are complete on surviving blast-exposed animals, they will be euthanized for collection of tissues, body fluids and blood.

### Example 6: Dosing Regime for Rodent Model

The SP is administered at the effective dose of 10 mg/kg CBD and 1 mg/Kg HU 211 via intraperitoneal (IP) injection 2 hours after exposure. This dose is repeated daily for 7 additional days.

### Example 7: Behavioral Tests on Rodent Model

### Sensorimotor Testing:

Spontaneous Forelimb Use: This test, described by Schallert and Lindner (Can J Psychol. 1990 44(2):276-92), assesses forelimb use during voluntary, spontaneous activity by evaluating the propensity of animals to adduct their forelimbs while rearing or standing. Animals are videotaped in a clear plastic cylinder for 5 minutes. The videotapes are scored in terms of forelimb-use asymmetry during vertical movements along the wall of the cylinder and for landings after a rear: (a) independent use of the left or right forelimb for contacting the wall of the cylinder during a full rear, to initiate a weight-shifting movement or to regain center of gravity while moving laterally in a vertical posture along the wall; wall lands/movements and floor lands are each expressed in terms of (a) percent use of the ipsilateral (non-impaired) forelimb relative to the total number of ipsilateral and contralateral placements. During a rear, the first limb to contact the wall with clear weight support (without the other limb contacting the wall within 0.5 seconds) is scored as an independent wall placement for that limb. Limb use ratio is calculated as

contralateral/(ipsilateral+contralateral).

### Cognitive Testing:

The analysis of cognitive function involves an assessment of spatial navigation using the water maze. Experiments that are primarily directed at assessing the activity of animals at numerous time points following TBI (such as when assessing the efficacy of therapeutic treatments designed to lessen the consequences of TBI) rely primarily on “acquisition” paradigms involving the simple place task and working memory task, in which the animals are required to learn a new platform location during each test session. This protocol does not involve pretraining or testing in the water maze prior to surgery.

The water maze used is a round pool (122 cm diameter; 60 cm deep) filled with water at 25° C., and rendered opaque by adding two pounds of white, non-toxic paint. The maze is located in a quiet, windowless room, with a variety of distinct, extramaze cues. Four points on the rim designated as north (N), east (E), south (S), and west (W), serve as starting positions and divide the maze into four quadrants. A round platform (10 cm in diameter) is placed 1.5 cm beneath the surface of the water, at a location that varies according to the requirements of the task. The animal's movement is videotaped with a CCD video which records the swim path. This animal's swim path is then analyzed with the Ethovision (Noldus) software program. This program determines path length, latency to reach the platform (in seconds), time spent in each quadrant of the water maze and swim speed.

The platform is located in the northeast quadrant of the maze. Each animal receives four 60 second trials each day. If the rat successfully locates the platform it is allowed to remain for 10 seconds; otherwise, it is put on the platform for a period of 10 seconds. Inter-trial intervals are two to four minutes, during which rats are placed under a heat lamp. Animals are tested after sensorimotor testing.

The probe trial consists of removing the platform and releasing the animal from the west position and videotaping the animal's swim pattern for 60 seconds. An animal that is not impaired should spend most of the time swimming in the quadrant that previously contained the hidden platform.

For the working memory task, the animal is given 60 seconds to find a submerged (non-cued) platform. If the rat fails to find the platform within 60 seconds, the animal is placed on the platform for 10 seconds. Five seconds following trial one for the same rat, a second identical trial is conducted. Rats are placed under a heat lamp for 4 minutes between each paired trial. After running the group of rats as above, the platform is moved to the next location of the maze and the procedure is repeated with this location. Five paired trials are given for each rat on each day.

A novel object recognition task is conducted in an open field arena with two different kinds of objects. Both objects will be consistent in height and volume, but different in shape and appearance. Animals are allowed to explore an empty arena and during habituation, the animals are exposed to this familiar arena with two identical objects placed at an equal distance. The following day the animals explore the open field in the presence of the familiar object and a novel object to test long-term recognition memory. The time spent exploring each object and the discrimination index percentage is recorded.

### Example 8: Hearing and Balance Tests on Rodent Model

### Auditory Brainstem Response (ABR):

Hearing thresholds are determined by auditory brainstem response via subcutaneous platinum needle electrodes placed at the vertex (reference), right mastoid (negative) and the left hind limb. Digitally generated stimuli consist of 1024 specific frequency tone bursts at between 3 and 30 kHz with a trapezoid envelop of 5 ms overall duration. The trapezoid is presented at a 3 ms plateau with 1 ms rise and fall. The stimulus is routed through a computer-controlled attenuator to an insert earphone (Etymotic Research ER-2). The sound delivery tube of the insert earphone is positioned about 5 mm from the tympanic membrane. The output of the insert earphone is calibrated by measuring the sound pressure level at a position 4-5 mm away from the tympanic membrane. Animals are placed in a plastic restraint tube during the forty-five minute recording procedure. The electrical response from the recording electrode is amplified (100,000×), filtered (100-3000 Hz) and fed to an A/D converter on a signal processing board in the computer. Eight hundred to twelve hundred samples are averaged at each level. Stimuli is presented at the rate of 16/second and the stimulus level is varied in 10 dB descending steps, until threshold is reached, then a 5 dB ascending step to confirm. Threshold is defined as the mid-point between the lowest level at which a clear response is seen and the next lower level where no response is seen. ABR is determined as a reproducible wave II response.

### Vestibular Evoked Myogenic Potentials (VEMP):

Vestibular Myogenic Potentials (cVEMP) to test balance function are measured before trauma and at multiple time points post-trauma, up to 30 days. The measurements are made with subcutaneous needle electrodes attached to a pre-amplifier and a data acquisition system (Intelligent Hearing Systems). Vestibular evoked myogenic potentials (VEMP) are measured by subdermal electrodes placed in neck muscles in response to acoustic stimuli that stimulate the saccule. Following the recordings, animals are observed for every 15 minutes for recovery from the anesthesia and once ambulatory once every hour until they are fully recovered from the anesthesia and are behaving normally. The residual function in non-treated animals is compared with treated animals. Potential side effects such as balance problems, rotating behaviors and pain is monitored. In acute studies, animals are euthanized at 24, 48 and 72 hours (immunohistochemical and gene expression) or at the conclusion chronic testing (histological analysis). From these studies, critical time for intervention, dose-response and optimal duration of treatment is established.

### Example 9: Histopathology in Rodent Model

### Tissue Harvesting:

After behavioral testing, animals are euthanized and tissues are collected in the following ways:

For histology, under overdose of ketamine (150 mg/kg) and xylazine (10 mg/kg) or isoflurane anesthesia, rats are transcardially perfused with saline followed by 4% paraformaldehyde. The head, liver and kidneys are removed and post fixed in the same fixative. Either decalcified heads or extracted brains are used for histopathological analysis.

For Mass Spectrometric Imaging, under overdose of ketamine (150 mg/kg) and xylazine (10 mg/kg) or isoflurane anesthesia, rats are transcardially perfused with saline to clear the blood. The brains are then removed rapidly and frozen in isopentane cooled with solid CO₂ (dry ice).

For molecular biology, under overdose of ketamine (150 mg/kg) and xylazine (10 mg/kg) or isoflurane anesthesia, rat are decapitated, tissue such as brain and kidney/liver is quickly removed and frozen in liquid nitrogen, and stored in −80° C. until use.

### Tissue Processing Histopathology:

After appropriate survival times, sham and traumatically exposed rats are anesthetized with sodium pentobarbital (100 mg/kg, i.p.) and perfused transcardially with 0.1 M PBS, followed by paraformaldehyde-lysine-periodate fixative. The intact heads are post-fixed in 4% paraformaldehyde for 24 hours at room temperature, decalcified in 10% formic acid to chemical 1 testing criterion and neutralized in overnight 5% sodium sulfate. After embedding in paraffin, sections are cut at 8-10 m in the horizontal plane. Every 25^{th} section is stained with hematoxylin and eosin for standard histopathological analysis. For immunohistochemistry, sets of sections are incubated sequentially in the following solutions at room temperature: 5% normal donkey serum (NDS) in 0.1 M PBS for 2 hours; primary antisera in 0.1 M PBS for 72 hours and 0.1 M PBS for 15 minutes. The antibodies are then visualized with either ABC peroxidase or an immunofluorescence method. Primary antibodies employed in decalcified heads with blast exposure include both neuronal and non-neuronal markers such as superoxide dismutase 2, interleukin 8 receptor, chemokine CXC motif receptor 3 angiopoietin 1, Vascular Endothelial Growth Factor A, TNF-alpha, and matrix metalloproteinase 2.

### Mass Spectrometric Imaging:

The animals are euthanized 1, 3 or 7 days post injury. Under ketamine/xylazine (100 mg/kg; 10 mg/kg) anesthesia, the chest of each rat is opened and the head perfused through a catheter placed in the ascending aorta with 50 to 100 ml of phosphate buffered saline at room temperature, allowing blood to flush from the head through an opening in the superior vena cava. When the perfusate is largely clear of blood, the skull is carefully opened and the brain dissected. After removing meninges, each brain is rapidly frozen in a small beaker containing about 30 ml of cold isopentane pre-cooled by immersion of the beaker in solid CO₂, then removed, wrapped individually in aluminum foil and stored at −80° C. until sectioned. The brains are sectioned in the coronal plane at a thickness of 18 microns using a cryostat (Leica Microsystems CM3050S, Bannockburn, Ill.). Tissue sections are implanted with silver nanoparticles (AgNP) 6 nm in diameter, using a nanoparticle implanter (Ionwerks, Houston, Tex.). A Thermo Scientific MALDI LTQ-XL-Orbitrap (Thermo Fisher Scientific, San Jose, Calif.) and Xcalibur software are used for used for matrix assisted laser desorption (MALDI) mass spectrometry imaging (MSI) data acquisition. Images of coronal sections are constructed from data collected in positive and negative ion mode, using a custom software package (Ionwerks, Houston, Tex.), which exports MS peak data for further statistical analysis in MATLAB.

1: A method for treating traumatic brain injury in a subject suffering therefrom, said method comprising administering to the subject a first composition comprising a N-Methyl-D-aspartate (NMDA) receptor antagonist and a second composition comprising a CB2 agonist, an agent which effectively increases an endogenous CB2 agonist or an agent which modifies levels of anandamide (AEA) or 2-arachidonoyl glycerol (2-AG).

2: The method of claim 1 wherein the first composition comprises a noncompetitive NMDA receptor antagonist.

3: The method of claim 1 wherein the first composition comprises 7-hydroxy-delta6-tetrahydrocannabinol 1,1-dimethylheptyl.

4: The method of claim 1 wherein the first composition and/or second composition is administered within 3-12 hours of the traumatic brain injury.

5-7. (canceled)

8: The method of claim 1 wherein the first composition is administered as a single dose or as multiple doses.

9. (canceled)

10: The method of claim 8 wherein the multiple doses are administered over a 72 hour period following the traumatic brain injury.

11: The method of claim 1 wherein the second composition comprises a non-cannabinoid CB2 agonist.

12: The method of claim 1 wherein the second composition comprises a cannabinoid CB2 agonist that also binds to an NMDA receptor.

13: The method of claim 1 wherein the second composition comprises an agent which increases levels of AEA.

14: The method of claim 1 wherein the second composition comprises an agent that decreases levels of 2-AG.

15: The method of claim 1 wherein the second composition comprises an inhibitor of fatty acid amide hydrolase.

16: The method of claim 1 wherein the second composition is administered 12 to 72 hours following the traumatic brain injury.

17: The method of claim 1 wherein the second composition is administered daily for up to 7 days following the traumatic brain injury.

18: The method of claim 1 wherein the first composition and/or the second composition is administered daily or every other day until symptoms of the traumatic brain injury are alleviated.

19: A method for treating traumatic brain injury in a subject suffering therefrom, said method comprising administering to the subject a first composition comprising an N-Methyl-D-aspartate (NMDA) receptor antagonist and a second composition comprising an anti-inflammatory agent capable of crossing the blood brain barrier.

20: The method of claim 1 wherein the second composition is administered before, simultaneously or after administration of the first composition.

21: The method of claim 19 wherein the second composition is administered before, simultaneously or after administration of the first composition.

22: The method of claim 1 wherein the traumatic brain injury treated comprises concussion.

23: The method of claim 19 wherein the traumatic brain injury treated comprises concussion.

24: A pharmaceutical composition for treatment of traumatic brain injury, said composition comprising:
a N-Methyl-D-aspartate (NMDA) receptor antagonist, a CB2 agonist, an agent which effectively increases an endogenous CB2 agonist, an agent which modifies levels of anandamide (AEA) or 2-arachidonoyl glycerol (2-AG), or an anti-inflammatory agent capable of crossing the blood brain barrier; and
a pharmaceutically acceptable vehicle.

25. (canceled)
